(19) United States
(12) Patent Application Publication
(10) Pub. No.: US 2025/0318482 A1
(43) Pub. Date: Oct. 16, 2025

(54) ZmCCD8 GENE-BASED MAIZE BREEDING METHODS

(71) Applicant: CHINA AGRICULTURAL UNIVERSITY, Beijing (CN)

(72) Inventors: Xuexian LI, Beijing (CN); Yanting ZHONG, Beijing (CN); Yongqi WANG, Beijing (CN)

(73) Assignee: CHINA AGRICULTURAL UNIVERSITY, Beijing (CN)

(21) Appl. No.: 19/177,446

(22) Filed: Apr. 11, 2025

(30) Foreign Application Priority Data
Apr. 11, 2024 (CN) 202410436173.1

(51) Int. Cl. A01H 1/00; A01H 1/04

(52) U.S. Cl. CPC A01H 1/101; A01H 1/045

(57) ABSTRACT

A use of a ZmCCD8 gene in maize breeding, including overexpressing the ZmCCD8 gene in maize plants through breeding to increase iron content in maize leaves and/or kernels. The ZmCCD8 gene has a nucleotide sequence as shown in SEQ ID NO: 1. The use of the ZmCCD8 gene in maize breeding can provide important genetic resources and technical support for improving crop quality and enhancing stress resistance, having important application prospects.

### CROSS-REFERENCE TO RELATED APPLICATIONS

This application claims priority to the Chinese patent application No. 2024104361731 filed on Apr. 11, 2024, the entire contents of which are hereby incorporated by reference.

### SEQUENCE LISTING

The instant application contains a Sequence Listing which is submitted electronically in XML format and is hereby incorporated by reference in its entirety. The XML copy, created on Jun. 11, 2025, is named “2025-06-11-Sequence Listing-65627-H038US00,” and is 9,693 bytes in size.

### TECHNICAL FIELD

The present disclosure relates to the field of molecular biology technology, and in particular, to a use of a ZmCCD8 gene in maize breeding, especially to a use of the ZmCCD8 gene in regulating the transport and accumulation of iron in leaves and kernels of maize plants, as well as a use of the ZmCCD8 gene in regulating the interaction between a ZmMYB118 protein and the promoter of ZmVIT2.1 in the plants.

### BACKGROUND

Iron (Fe) is an essential trace element for both animal and plant growth. In humans and animals, iron plays a vital role in blood cell formation and overall development. Iron deficiency in humans can lead to anemia. For plants, iron is indispensable for key cellular activities like respiration, photosynthesis, and chlorophyll synthesis. Although iron is plentiful in Earth's crust, it primarily exists in the form of ferric iron (Fe³⁺) that can be barely absorbed by plants. Especially, in alkaline soils, the low solubility makes it difficult to be absorbed and utilized, often resulting in iron deficiency in the plants. When the plants are iron-deficient, it causes yellowing between leaf veins (interveinal chlorosis) in new leaves, which in turn affects plant growth and development, ultimately reducing crop yield and quality.

To address iron deficiency in crops, farmers currently rely on management measures such as soil applications or foliar sprays of iron sulfate (FeSO₄) and chelated iron fertilizers. However, these measures often cost much more and fail to provide an efficient long-term solution. For severely iron-deficient soils, particularly alkaline and high-carbonate soils, researchers are now prioritizing studies on the physiological and molecular mechanisms governing iron uptake, transport, and storage in plants. By combining approaches in life sciences such as plant physiology, molecular biology, and genetics, this emerging field has become a key research focus for improving agricultural productivity and food security.

Studies show that iron is absorbed into stele by plant roots and then loaded into vascular bundles for long-distance transport to young tissues or kernels. This process involves the regulation of numerous transport proteins.

It has been reported that in rice, Zinc/Iron-regulated transporter-like Protein (ZIP) family genes OsIRT1 and OsIRT2 function as iron transporters, regulating iron uptake by plant roots. Similar findings have been reported in maize, for example, the ZIP family genes ZmIRT2 and ZmZIP5 are involved in iron uptake by the root. In addition, ZmZIP5 facilitates the transport of iron from the root to the shoot.

It has also been reported that members of the Yellow Stripe1-Like (YSL) family are involved in iron transport processes. In rice, YSL2 transports iron in the form of a nicotinamide chelate. In maize, YSL2 mediates endosperm-to-embryo allocation of iron, thereby regulating iron homeostasis in kernels. In Arabidopsis, a vacuolar membrane transporter VIT1 affects plant growth by regulating iron accumulation within the vacuole. In rice, VIT1 and VIT2 are highly expressed in flag leaves and leaf sheaths, and their function loss results in increased iron content in grains and decreased iron levels in the flag leaf, indicating that they regulate flag leaf-to-grain transport of iron. Under conditions of iron excess, OsVIT2 also affects iron concentrations in both brown and polished rice. However, the role of VIT transporters in regulating iron transport and storage in maize remains unexplored.

The ancient Carotenoid Cleavage Dioxygenase (CCD) gene family is widely present across bacteria, fungi, plants, and animals. CCD family enzymes cleave carbon-carbon double bonds of carotenoids to generate apocarotenoids such as abscisic acid and strigolactone, which are widely involved in plant growth and development. These enzymes depend on non-heme ferrous iron for catalytic activities, utilizing four strictly conserved histidine residues and three coordinating glutamates to bind both iron and oxygen. In plants, CCD7 and CCD8 sequentially cleave carotenoids to generate carlactone, which is then converted into strigolactone by the cytochrome enzyme P450. Strigolactones are perceived by the receptor DWARF 14 (D14), which recruits leucine-rich repeat-containing F-box protein D3 to degrade strigolactone suppressor D53, regulating the expression of downstream transcription factors and affecting the growth and development of crops.

As a key strigolactone biosynthetic enzyme, CCD8 critically regulates root architecture, shoot branching, and fruit development. The loss-of-function mutants of MAX4/D10b, CCD8 homologous genes in Arabidopsis and rice, respectively, exhibit significant multi-branched phenotypes. In maize, CCD8 mutation significantly inhibits both root and shoot development. Furthermore, CCD8 regulates kernel size through modifying maternal pedicel tissues during maize domestication.

Most research on the CCD8 focuses on its involvement in the processes of phosphorus and nitrogen uptake and utilization. Under low phosphorus, the expression of CCD8 is up-regulated in roots, boosting strigolactone biosynthesis, thereby promoting mycorrhizal symbiosis to participate in plant phosphorus absorption and distribution. CCD8 also regulates nitrogen uptake by influencing root nodulation. However, its role in regulating accumulation of the micronutrient (iron) in plants remains unknown.

As a staple crop, enhancing iron content in maize kernels could significantly address human iron needs. Iron distribution in maize kernels shows the highest concentrations in scutellum, embryo, and aleurone layers, and minimal levels in endosperm. The iron content in maize kernels typically ranges from 11.3 to 60 mg/kg, while most conventional varieties consistently fall at the lower end of this range. With rising challenges from high-pH and high-carbonate soils, maintaining the current iron content in corn kernels has become an urgent agricultural priority.

Therefore, there is a need to provide a use of a ZmCCD8 gene in maize breeding and the related method. By identifying functional genes that regulate plant iron homeostasis and elucidating its function and biological regulatory mechanisms, it is of great theoretical and practical significance to maintain or even increase the iron content in maize kernels.

### SUMMARY

One or more embodiments of the present disclosure provide a use of a ZmCCD8 gene in maize breeding, comprising: overexpressing the ZmCCD8 gene in maize plants through breeding to increase iron content in maize leaves and/or kernels, wherein the ZmCCD8 gene has a nucleotide sequence as shown in SEQ ID NO: 1.

In some embodiments, the ZmCCD8 gene encodes a protein having an amino acid sequence as shown in SEQ ID NO: 2.

In some embodiments, overexpressing the ZmCCD8 gene in the maize plants increases the iron content in ear leaves.

In some embodiments, overexpressing the ZmCCD8 gene in the maize plants increases the iron content in kernels per ear.

One or more embodiments of the present disclosure provide a use of a ZmCCD8 gene in regulating interaction between a ZmMYB118 protein and a promoter of ZmVIT2.1, comprising: overexpressing the ZmCCD8 gene in maize plants through breeding to regulate the interaction between the ZmMYB118 protein and the promoter of ZmVIT2.1; wherein the ZmCCD8 gene has a nucleotide sequence as shown in SEQ ID NO: 1, the ZmMYB118 protein is encoded by a nucleotide sequence as shown in Zm00001d032024 from the Phytozome database, and the promoter of ZmVIT2.1 has a nucleotide sequence as shown in Zm00001d005715 from the Phytozome database.

One or more embodiments of the present disclosure provide a method for maize breeding, comprising: overexpressing a ZmCCD8 gene in maize plants to increase iron content in leaves and/or kernels; and obtaining a maize inbred line overexpressing the ZmCCD8 gene; wherein the ZmCCD8 gene has a nucleotide sequence as shown in SEQ ID NO: 1.

### BRIEF DESCRIPTION OF THE DRAWINGS

The present disclosure will be further illustrated by way of exemplary embodiments, which will be described in detail through the accompanying drawings, wherein:

FIG. 1 is a schematic diagram of two ZmCCD8 knockout (KO) lines according to some embodiments of the present disclosure;

FIG. 2 is a schematic diagram illustrating relative expression levels of the ZmCCD8 gene in two ZmCCD8 overexpressed (OE) lines according to some embodiments of the present disclosure;

FIG. 3 is a schematic diagram illustrating phenotypic comparison of V8-stage newly expanded leaves and mature ears between the ZmCCD8 KO and OE lines according to some embodiments of the present disclosure;

FIG. 4 is a schematic diagram illustrating Soil and Plant Analyzer Development values (SPAD value, characterizing the chlorophyll content of leaves) of V8-stage newly expanded leaves in the ZmCCD8 KO and OE lines according to some embodiments of the present disclosure;

FIG. 5 is a schematic diagram illustrating the kernel weight at maturity in the ZmCCD8 KO and OE lines according to some embodiments of the present disclosure;

FIG. 6 is a schematic diagram illustrating iron content in ear leaves at maturity in the ZmCCD8 KO and OE lines according to some embodiments of the present disclosure;

FIG. 7 is a schematic diagram illustrating iron content in kernels at maturity in the ZmCCD8 KO and OE lines according to some embodiments of the present disclosure;

FIG. 8 is a schematic diagram illustrating a number of differentially expressed genes in the ZmCCD8 KO and OE lines according to some embodiments of the present disclosure;

FIG. 9 is a schematic diagram illustrating a pathway enrichment analysis of differentially expressed genes in the ZmCCD8 KO and OE lines according to some embodiments of the present disclosure;

FIG. 10 is a schematic diagram illustrating an analysis of differentially expressed genes related to the RNA process in the ZmCCD8 KO and OE lines according to some embodiments of the present disclosure;

FIG. 11 is a schematic diagram illustrating an analysis of differentially expressed genes related to iron transport in the ZmCCD8 KO and OE lines according to some embodiments of the present disclosure;

FIG. 12 is a schematic diagram illustrating relative expression levels of ZmMYB118 and ZmVIT2.1 in kernels 7 days after pollination in the ZmCCD8 KO and OE lines according to some embodiments of the present disclosure;

FIG. 13 is a schematic diagram illustrating a yeast one-hybrid assay of the binding of the ZmMYB118 protein and an MBS cis-acting element in the promoter region of ZmVIT2.1 according to some embodiments of the present disclosure;

FIG. 14 is a schematic diagram illustrating an electrophoretic mobility shift assay (EMSA) of the binding of the His-ZmMYB118 protein and an MBS cis-acting element in the promoter region of ZmVIT2.1 according to some embodiments of the present disclosure;

FIG. 15 is a schematic diagram illustrating the results of Chromatin Immunoprecipitation-quantitative Polymerase Chain Reaction (ChIP-qPCR) according to some embodiments of the present disclosure;

FIG. 16 is a schematic diagram illustrating the results of a dual-luciferase reporter assay according to some embodiments of the present disclosure; and

FIG. 17 is a schematic diagram illustrating the results of iron absorption activities of ZmVIT2.1 downstream of ZmCCD8 in a yeast system according to some embodiments of the present disclosure.

### DETAILED DESCRIPTION

To clarify the objectives, technical solutions, and advantages of the embodiment of the present disclosure, the technical solutions in the embodiments of the present disclosure are illustrated by the following detailed description of embodiments in the disclosure. Note that these embodiments represent only a subset of possible embodiments, not an exhaustive compilation. Furthermore, any additional implementations derivable from these embodiments by practitioners skilled in the art, without requiring inventive steps, shall naturally fall within the scope of protection of the present disclosure.

One or more embodiments of the present disclosure provide a use and a related method of the ZmCCD8 gene in maize breeding.

A nucleotide sequence of the ZmCCD8 gene is as shown in Zm00001d043442 from the Phytozome database. The ZmCCD8 gene from different maize inbred lines or varieties may exhibit nucleotide sequence variations, but any gene exhibiting at least 80% sequence identity (such as 85%, 90%, 95%, 98%, 99%, or even 100% sequence identity) with a wild-type ZmCCD8 gene and possessing at least one of biological functions described herein falls within a scope of the present disclosure. These biological functions may include: (1) increasing iron content in leaves, particularly in ear leaves; (2) enhancing iron content in kernels, particularly in kernels per ear; (3) promoting chlorophyll biosynthesis; and (4) inhibiting the interaction between the ZmMYB118 protein and the promoter of ZmVIT2.1 in the plant. However, the biological functions are not limited to the above and may also include other related functions, such as boosting maize yield, particularly the yield of maize grown in alkaline soils.

In some embodiments, the ZmCCD8 gene has a nucleotide sequence as shown in SEQ ID NO: 1.

In some embodiments, the ZmCCD8 gene encodes a protein having an amino acid sequence as shown in SEQ ID NO: 2.

One or more embodiments of the present disclosure provide a use of the ZmCCD8 gene in promoting plant growth. In some embodiments, overexpression of ZmCCD8 in the plants may promote plant growth.

One or more embodiments of the present disclosure provide a use of the ZmCCD8 gene in increasing maize yield, particularly in enhancing kernel weight per ear. In some embodiments, overexpression of ZmCCD8 in plants may increase grain yield, such as improving maize kernel weight per ear.

One or more embodiments of the present disclosure provide a use and a related method of the ZmCCD8 gene in increasing iron content in maize leaves, particularly in ear leaves. In some embodiments, overexpression of ZmCCD8 in the plants may increase iron content in leaves, especially in ear leaves.

One or more embodiments of the present disclosure provide a use of the ZmCCD8 gene in enhancing iron content in maize kernels, especially in kernels per ear. In some embodiments, overexpression of the ZmCCD8 in the plants may increase iron content in maize kernels, especially in kernels per ear.

One or more embodiments of the present disclosure provide a use and a related method of the ZmCCD8 gene in regulating interaction between the ZmMYB118 protein and the promoter of ZmVIT2.1 in plants. In some embodiments, overexpression of the ZmCCD8 gene may inhibit interaction between the ZmMYB118 protein and the promoter of ZmVIT2.1 in the plants. Therefore, by modulating the expression of the ZmCCD8 gene or the activity of its functional protein, it is possible to regulate the interaction between the ZmMYB118 protein and the promoter of ZmVIT2.1, thereby influencing the biological functions associated with ZmMYB118 and ZmVIT2.1 pathways. The amino acid sequences of the ZmMYB118 protein and the ZmVIT2.1 protein and the nucleotide sequences encoding the ZmMYB118 protein and the ZmVIT2.1 protein are known. The amino acid sequence of the ZmMYB118 protein and the nucleotide sequence encoding the ZmMYB118 protein may be seen in Zm00001d032024 from the Phytozome database. The amino acid sequence of the ZmVIT2.1 protein and the nucleotide sequence encoding the ZmVIT2.1 protein may be seen in Zm00001d005715 from the Phytozome database.

As used herein, “overexpression” refers to the generally accepted meaning in the art, namely, a higher expression level of the ZmCCD8 gene in genetically engineered plants compared to wild-type plants. Such overexpression may occur at a transcriptional level, a translational level, or both.

One or more embodiments of the present disclosure provide a method for maize breeding, comprising: overexpressing the ZmCCD8 gene in maize plants to increase iron content in maize leaves and/or kernels; and obtaining a maize inbred line overexpressing the ZmCCD8 gene. Genetic engineering techniques known in the art may be used to achieve overexpression of the ZmCCD8 gene.

One or more embodiments of the present disclosure provide a maize inbred line characterized by overexpression of the ZmCCD8 gene. In some embodiments, the maize inbred line may be obtained using the breeding method described above. In some embodiments, the maize inbred line may be obtained by overexpression of the ZmCCD8 gene in the maize plants through genetic engineering techniques. In some embodiments, the maize inbred line exhibits at least one of the following phenotypes resulting from ZmCCD8 gene overexpression: (1) increasing iron content in leaves, particularly in ear leaves; (2) enhancing iron content in kernels, especially in kernels per ear; (3) promoting chlorophyll biosynthesis; (4) inhibition of the interaction between the ZmMYB118 protein and the promoter of ZmVIT2.1.

In some embodiments, comparison between the ZmCCD8 knockout and overexpression lines reveals that the gene significantly influences iron content in leaves (e.g., ear leaves at maturity) and kernels. Compared to the wild-type line, the iron content in ear leaves of the ZmCCD8 knockout lines is significantly reduced, whereas the iron content in ear leaves of the ZmCCD8 overexpression lines is significantly increased. The iron content in kernels per ear of the ZmCCD8 knockout lines is significantly reduced, and the phenotype of the ZmCCD8 knockout lines is opposite to that of the ZmCCD8 overexpression lines. In seedling shoots and kernels, ZmCCD8 overexpression is found to suppress the expression of ZmMYB118 and ZmVIT2.1, and the ZmMYB118 protein binds to the promoter of ZmVIT2.1. These findings indicate that the ZmCCD8 gene positively regulates iron accumulation in leaves, particularly in ear leaves and kernels. The function of the ZmCCD8 gene provides a valuable genetic resource for iron transport and accumulation in crops, particularly in maize, and offers crucial genetic resources for breeding crop varieties (especially maize) with high iron content, thereby addressing the iron deficiency challenges associated with growing maize in alkaline soils from the perspective of breeding.

The following examples are provided to further illustrate the present disclosure, but are not intended to limit the scope of protection. Unless otherwise specified, the experimental methods used in the following examples are conventional methods known to those skilled in the art.

### EXAMPLES

### Example 1 Generation of ZmCCD8 Knockout and Overexpression Lines

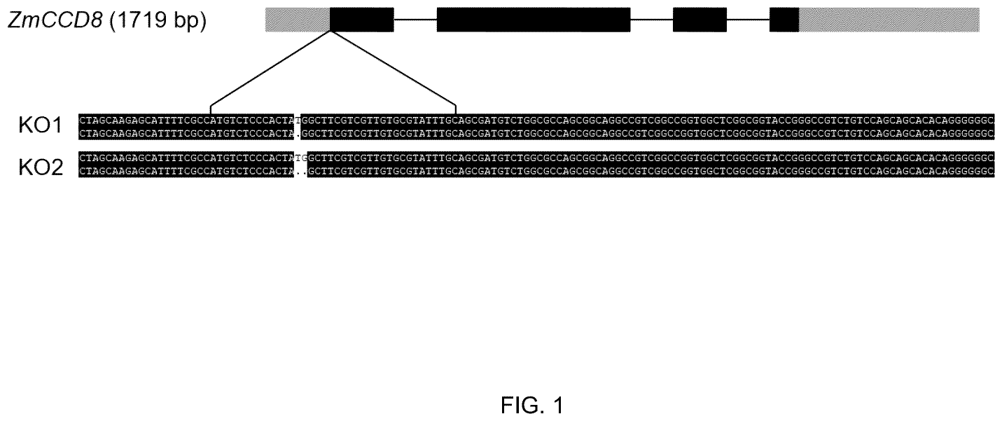

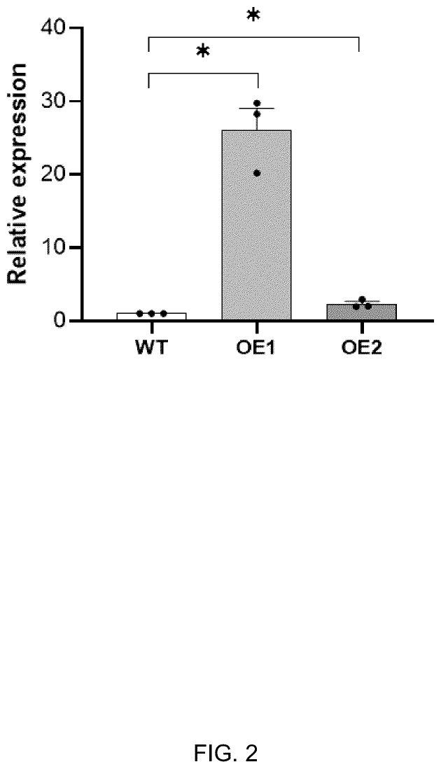

(1) In the example, the ZmCCD8 gene has a nucleotide sequence as shown in SEQ ID NO: 1, and the ZmCCD8 gene encodes a protein having an amino acid sequence as shown in SEQ ID NO: 2. The CRISPR/Cas9 edited mutant of the ZmCCD8 gene was purchased from Beijing Bomei Xingao Technology Co., Ltd. A vector pCAMB3301 carrying the sgRNA of ZmCCD8 was used to transform a maize inbred line B104 through the following procedure: first, the constructed vector carrying the sgRNA was transformed into Agrobacterium EHA105, then the maize immature embryos were transformed by the Agrobacterium-mediated method, and finally, the positive transformed plants were screened by sequencing to obtain two distinct ZmCCD8 knockout lines (as shown in FIG. 1), named KO1 (a T deletion at position 28 after the start codon of the ZmCCD8 gene) and KO2 (a TG deletion at positions 28 and 29 after the start codon of the ZmCCD8 gene).
(2) The overexpression lines of the ZmCCD8 gene used in the present disclosure were obtained by transforming the maize inbred line B104 with a vector pCAMB1300. The full-length coding sequence (CDS) of the ZmCCD8 gene was first cloned into the expression vector pCAMB3301 with a CaMV 35S promoter to generate a recombinant vector. The recombinant vector was then introduced into Agrobacterium EHA105, and maize immature embryos were transformed by the Agrobacterium-mediated method. Positive transformed plants were identified with screening using glufosinate herbicide.
(3) After identifying the positive transformed plants, total RNA was extracted from the wild-type (WT) line, the ZmCCD8 knockout lines, and the ZmCCD8 overexpression lines using Trizol reagent. RNA sample concentrations were measured using a NanoDrop spectrophotometer, and the RNA samples were then reverse transcribed using the PrimeScript™ RT reagent Kit (Perfect Real Time) from Takara Bio Inc. (Beijing). In addition, through RT-qPCR analysis, two overexpression lines with significantly increased ZmCCD8 gene expression (relative expression) levels were identified and designated as OE1 and OE2 (as shown in FIG. 2). The RT-qPCR was performed using Takara's TB Green® Premix Ex Taq™ II kit, with the maize ubiquitin (UBI) gene as the internal reference.

### Example 2: Phenotypic Analysis of ZmCCD8 Knockout and Overexpression Lines

(1) Homozygous seeds from the wild-type line, the ZmCCD8 knockout lines, and the ZmCCD8 overexpression lines were sown at the Langfang transgenic experimental base. Prior to sowing, basal fertilizers were applied with 60 kg/ha of urea, 135 kg/ha of calcium superphosphate, and 80 kg/ha of potassium sulfate. Subsequently, additional fertilizers were applied at an 8-leaf stage with 120 kg/ha of urea and at a 12-leaf stage with 70 kg/ha of urea and 40 kg/ha of potassium sulfate. When the 8th leaf was fully expanded (V8 stage), the phenotype of this newly expanded leaf was recorded and a Soil and Plant Analyzer Development (SPAD) value of the new leaf was measured. At maturity, maize ears were photographed and the kernel weight per ear was weighed.

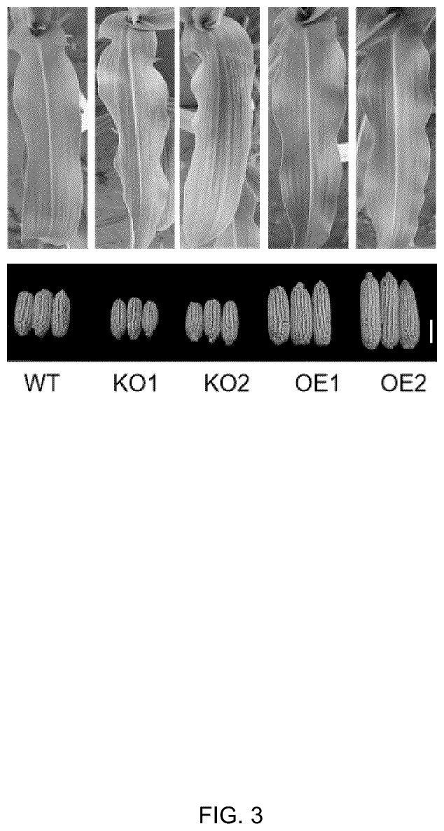

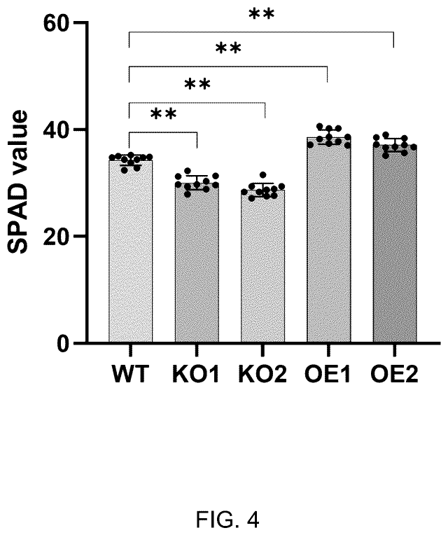

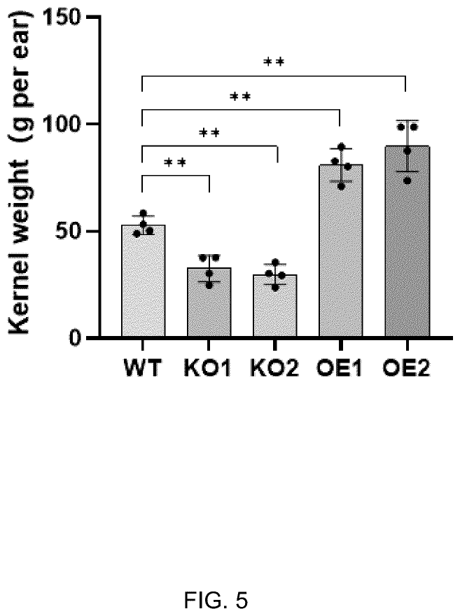

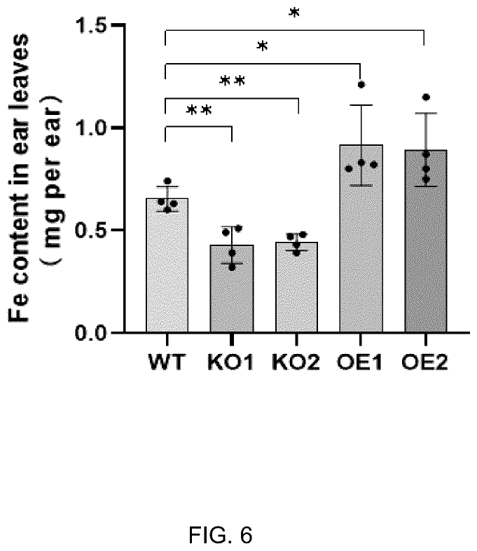

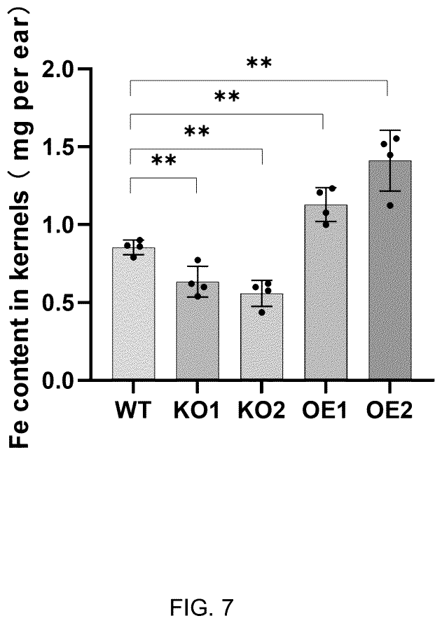

At the V8 stage, compared to the wild-type line, the ZmCCD8 knockout lines exhibit interveinal chlorosis on the new leaves, while new leaves of the ZmCCD8 overexpression lines remain uniformly green. The SPAD value of the new leaves in the ZmCCD8 knockout lines is lower than that of the wild-type line, whereas the ZmCCD8 overexpression lines exhibit the opposite pattern (as shown in FIGS. 3 and 4). Further analysis of kernel weight at maturity reveals that the kernel weight of the ZmCCD8 knockout lines is significantly lower than the wild-type line, while the kernel weight of the ZmCCD8 overexpression lines is significantly higher than the wild-type lines (as shown in FIG. 5).
(2) After digestion of ear leaves and kernels at maturity using the HNO₃—H₂O₂ digestion method in a microwave digestion instrument (CEM, Matthews, NC, USA), iron content in the ear leaves and kernels was measured by an inductively coupled plasma optical emission spectroscopy (ICP-OES, OPTIMA 3300DV, Perkin Elmer, USA). The results indicate that iron content in both the ear leaves and kernels of the ZmCCD8 knockout lines is significantly lower than that in the wild-type line, while the iron content in the ear leaves and kernels of the ZmCCD8 overexpression lines is significantly higher than in the wild-type line (as shown in FIGS. 6 and 7). These results suggest that the ZmCCD8 gene regulates the accumulation of iron in maize leaves and kernels at maturity.

### Example 3: Transcriptome Analysis of the ZmCCD8 Knockout and Overexpression Lines at the Seedling Stage

Homozygous seeds from the wild-type line, the ZmCCD8 knockout line, and the ZmCCD8 overexpression line were first disinfected with 10% hydrogen peroxide for 30 minutes, followed by five washes with deionized water. The seeds were then soaked in saturated calcium sulfate for 6 hours, washed five times with deionized water, and spread out on germination trays for dark treatment for 2-4 days to promote sprouting. Once the primary root reached 1-2 cm in length, the seedlings were rolled with double-layer filter paper to maintain their upright posture. On day 10 after germination, the entire seedling was harvested for transcriptomic sequencing.

Total RNA was extracted using the Trizol reagent kit from TianGen Biotech Co., Ltd. (Beijing). RNA concentrations and purity were checked, and RNA integrity was assessed using the Agilent 2100 Bioanalyzer. The eukaryotic transcriptome was sequenced using the Illumina high-throughput sequencing platform. The raw data were filtered for quality control, and adapter sequences were removed to obtain high-quality data. This process was completed by Novogene Bioinformatics Technology Co., Ltd. (Beijing). The sequencing data were aligned to the V4 version of the maize B73 genome database using TopHat and Bowtie2 programs for gene annotation (ftp://ftp.ensemblgenomes.org/pub/plants/release-44/fasta/zea_mays). The Cufflinks program (version 2.2.1) was used to calculate the Fragments Per Kilobase of transcript per Million mapped reads (FPKM) value for each gene, and the P value of differential expression of a single gene between the transgenic and wild-type samples was calculated. The P value was corrected using a multiple-test correction method to obtain a false discovery rate (FDR) value. Differentially expressed genes (DEGs) were identified with a gene expression change greater than 2-fold and an FDR value less than 0.05 as thresholds. Pathway enrichment analysis was performed on DEGs using Mapman, and the results were visualized in Excel.

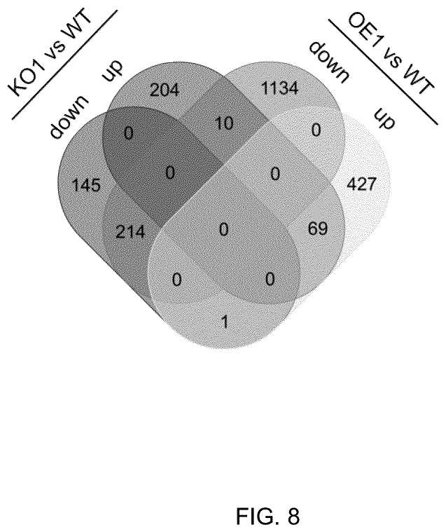

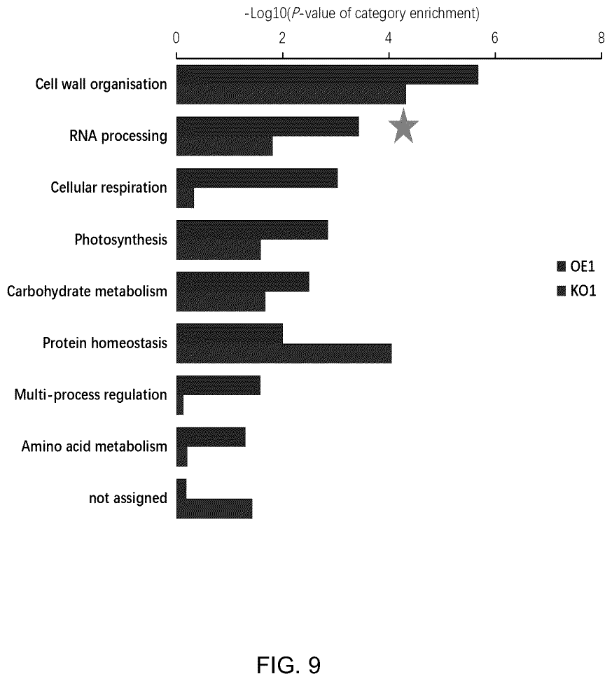

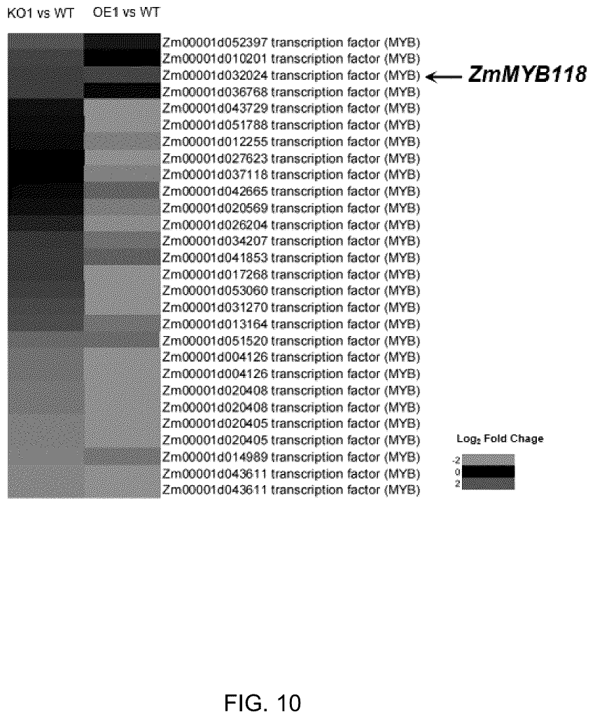

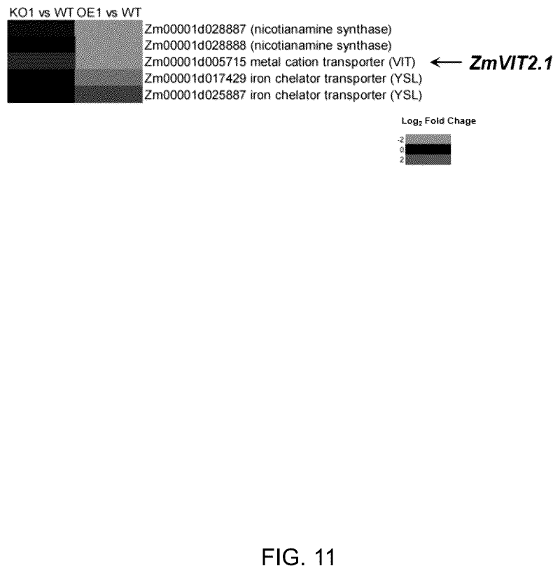

Transcriptomic analysis reveals that compared to the wild-type line, 643 DEGs were identified in the ZmCCD8 knockout line, and 1855 DEGs were identified in the ZmCCD8 overexpression line (as shown in FIG. 8). Pathway enrichment analysis indicates that these DEGs are primarily enriched in cell wall organization, RNA processing, and cellular respiration (as shown in FIG. 9). Further analysis of RNA processing-related DEGs indicates that a transcription factor ZmMYB118 (with nucleotide sequence as shown in the Zm00001d032024 from the Phytozome database) is found to have an opposite expression trend in the DEGs of the ZmCCD8 knockout and overexpression lines (as shown in FIG. 10). By analysis of the DEGs in both ZmCCD8 knockout and overexpression lines, five genes related to iron transport are identified, including ZmVIT2.1 (with a nucleotide sequence as shown in Zm00001d005715 from the Phytozome database) which encodes a vacuolar membrane iron transporter protein (as shown in FIG. 11). These results suggest that ZmMYB118 and ZmVIT2. 1 are downstream genes of ZmCCD8.

### Example 4: Detection of Relative Expression Levels of ZmMYB118 and ZmVIT2.1 in Kernels of ZmCCD8 Knockout and Overexpression Lines 7 Days After Pollination

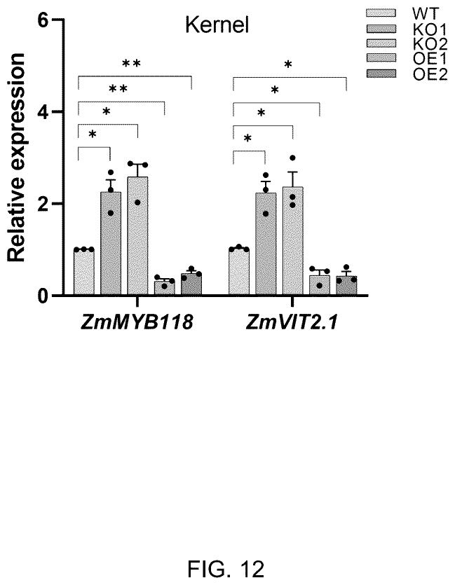

Following the method described in section (1) of Example 2, the homozygous seeds from the wild-type line, the ZmCCD8 knockout lines, and the ZmCCD8 overexpression lines were sown at the Langfang transgenic experimental base. Seven days after pollination, fresh kernel samples from the wild-type, ZmCCD8 knockout, and ZmCCD8 overexpression lines were collected and rapidly frozen in liquid nitrogen and stored at −80° C. RNA extraction and reverse transcription were performed according to the method described in section (3) of Example 1. The relative expression levels of ZmMYB118 and ZmVIT2.1 were detected using a fluorescence quantitative PCR instrument. The results indicate that compared to the wild-type line, ZmMYB118 and ZmVIT2.1 are upregulated in kernels of the ZmCCD8 knockout lines, while they are downregulated in the kernels of the ZmCCD8 overexpression lines (as shown in FIG. 12).

### Example 5: Analysis of Interaction and Regulation Between ZmMYB118 and ZmVIT2.1

### (1) Yeast One-Hybrid Assay

Vector construction: For the promoter region of ZmVIT2.1 (with the nucleotide sequence as shown in Zm00001d005715 from the Phytozome database), an approximately 500 bp sequence containing the MBS (TAACTG) or mutated MBS (AAAAAA) elements was cloned into the pLacZi 2u vector. The CDS of ZmMYB118 (with the nucleotide sequence as shown in Zm00001d032024 from the Phytozome database) was cloned into the pB42AD vector. The sequencing verification was performed.

Preparation and transformation of yeast competent cells: the EGY48 yeast strain was streaked on Yeast Extract Peptone Dextrose (YPD) plates, and the single colony was inoculated into the YPD liquid medium and shaking-cultured at 30° C. and 200 rpm until the optical density (OD600) value reached 1.5. The culture was then transferred to 250 mL of the YPD liquid medium and shaking-cultured for 3 hours at 30° C. and 200 rpm until the OD600 reached 0.4-0.6. Yeast cells were harvested by centrifugation at 4000 rpm for 5 minutes at room temperature and washed with sterilized ultrapure water. The cells were then resuspended in 1.5 mL of 1×TE/LiAc (10×TE:10×LiAc=1:1, v/v) and kept on ice. 10 μL of boiled salmon sperm DNA, 150 ng of plasmid, 100 μL of yeast competent cells, and 600 μL of PEG/TE/LiAc (50% PEG:10×TE:10×LiAc=8:1:1, v/v) were added into 1.5 mL sterile centrifuge tubes, shaken and incubated for 30 minutes, followed by the addition of DMSO to stop the reaction. After centrifuging at room temperature and discarding the supernatant, cells were resuspended in 200 μL of 1×TE solution and spread onto the SD/−Ura-Trp medium. After 3 days of incubation in a 30° C. constant-temperature incubator, single colonies were selected with a sterilized pipette tip and streaked in pairs on SD/−Ura-Trp+X-Gal medium for cultivation, and then photographed and recorded after the positive strain became blue with the substrate reaction.

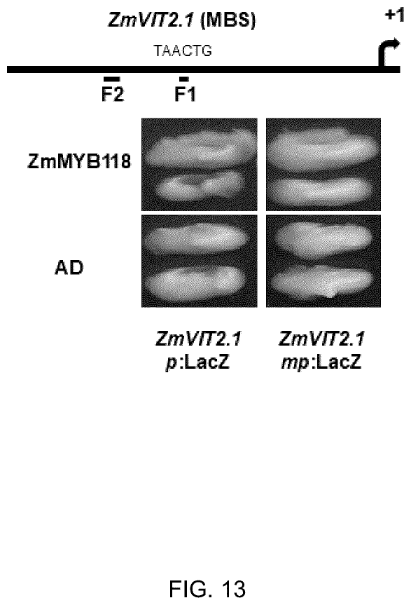

Analysis of the promoter region of ZmVIT2.1 reveals an MYB transcription factor cis-acting element (MBS: TAACTG) at positions 1362-1367 bp. By the yeast one-hybrid assay, it is found that co-transformation of ZmMYB118 and the ZmVIT2.1 promoter containing the MBS element into yeast cells results in blue yeast colonies, however, when ZmMYB118 and the ZmVIT2.1 promoter sequence containing the mutated MBS (AAAAAA) are co-transformed into the yeast cells, the yeast strain remains white, demonstrating that ZmMYB118 can bind to the MBS element in the promoter region of ZmVIT2.1 in the yeast system (as shown in FIG. 13).

### (2) Electrophoretic Mobility Shift Assay (EMSA)

Vector construction: the CDS of ZmMYB118 was cloned into the PET-30a expression vector and verified by sequencing to obtain a recombinant vector with the cloned CDS of ZmMYB118. Additionally, a 40-bp MBS-containing promoter region of ZmVIT2.1 was referred to as a probe sequence, and its biotin labeled and unlabeled versions were synthesized by Shanghai Jierui Company (the forward primer sequence was shown as in SEQ ID NO: 3; the reverse primer sequence was shown as in SEQ ID NO: 4).

Prokaryotic protein expression and purification: the recombinant vector (expressing the ZmMYB118 protein) was transformed into competent cells of E. coli BL21 (DE3) and cultured overnight at 37° C. Single colonies were picked and cultured in the LB liquid medium with Kan⁺ antibiotics at 37° C. and 200 rpm overnight. The culture was inoculated into 300 mL of the liquid medium and grown until OD600 reached 0.6-0.8. Protein expression was induced with 1% IPTG, and cells were harvested by centrifugation. After resuspending in the lysis buffer, the cells were broken to release the His-tagged ZmMYB118 protein (His-ZmMYB118) using the ultrasonic crushing instrument, and the protein was purified using nickel-containing magnetic agarose beads and stored at −80° C.

Following the instructions of the LightShift® Chemiluminescent EMSA Kit (Thermo Scientific), the following steps were performed. The probe sequences synthesized by the company were diluted with ddH₂O to 100 μM and the forward and reverse primers for each probe were added, respectively. The biotin-labeled probe sequences were then diluted to 0.2 μM, and the cold probe sequences were diluted to 10 μM (50×) and 20 μM (100×), and then placed in a PCR machine at 96° C. for 5 minutes to anneal. A non-denaturing polyacrylamide gel was prepared (5×TBE 1 mL, 30% acrylamide 2 mL, 50% glycerol 0.25 mL, ddH₂O 6.7 mL, TEMED 5 μL, 10% APS 75 μL) and pre-electrophoresed for 1 hour in the electrophoresis tank. Probes containing MBS and the His-ZmMYB118 protein, as well as a 6×His protein, were added to the reaction system, which was reacted in the PCR machine at 25° C. for 20 minutes. After the reaction, the loading buffer was added to each sample, mixed well, and electrophoresed at 100 V. Then, the membrane was transferred in an ice bath at 60 V for 40 minutes. After the transfer, the membrane was crosslinked for 1 minute using a UV crosslinker. The membrane was blocked with the blocking buffer and then horseradish peroxidase (HRP) was added. After washing with a washing buffer and equilibrating with a substrate buffer, the substrate reaction solution was added, and the membrane was developed and photographed.

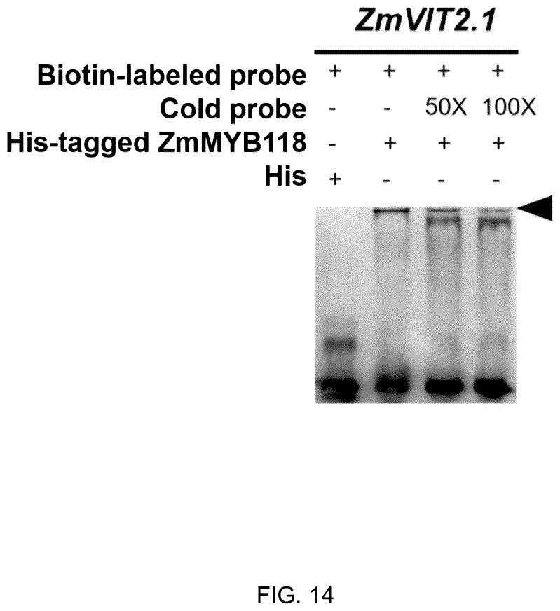

From the electrophoretic mobility shift assay, it is observed that the ZmMYB118 protein could bind to the biotin-labeled MBS oligonucleotide, affecting the mobility of the ZmMYB118 protein. As the concentration of the cold probe (non-biotin-labeled probe) increases, the signal from the biotin-labeled probe weakens, as shown in FIG. 14, indicating that ZmMYB118 can bind to the MBS cis-element in the promoter region of ZmVIT2.1 in vitro.

### (3) Chromatin Immunoprecipitation-Quantitative Polymerase Chain Reaction (ChIP-qPCR)

Vector construction: The stop codon is removed from the CDS of ZmMYB118 and the obtained CDS was ligated to the pUC19-35S-HA-RBS vector, and the obtained vector was confirmed by sequencing.

Preparation and transformation of protoplasts: Maize inbred line B73 was germinated and grown in darkness at 28° C. until two fully expanded leaves and one emerging leaf were visible. The well-growing leaves were quickly cut transversely and placed in the enzymatic hydrolysis solution. The solution was vacuumed for 30 minutes and incubated at 28° C. and 40 rpm for 3-4 hours. Protoplasts were filtered through a 300-mesh filter cloth, washed with a W5 solution (a commonly used wash buffer for plant protoplast isolation), and then centrifuged at 100 g with an acceleration of 1 and a deceleration of 0 for 2 minutes. The supernatant was discarded, and the protoplasts were resuspended in the cold W5 solution and stood on ice for 30 minutes. The protoplasts were then collected by centrifugation and resuspended in an Mmg solution (a commonly used buffer for plant protoplast transformation) and stood on ice for later use. 300 μg plasmid and 4 mL protoplast solution were added to a 50 mL Corning tube, respectively. After gently mixing, 4.3 mL 40% polyethylene glycol (PEG) was added to induce transformation, gently mixing, standing for 18 min, and adding 17.2 mL W5 solution to terminate the transformation. Each plasmid transformation was repeated three times. After centrifugation, the protoplasts were collected and resuspended in the W5 solution. By centrifugation again, the supernatant was discarded, and the protoplasts were resuspended gently in a WI solution (a commonly used buffer for protoplast separation, transformation, or culture) and incubated at 28° C. in the dark for 14-18 hours. The protoplasts were then collected by centrifugation, the supernatant was discarded, and immunoprecipitation was performed after sample cross-linking, nuclear lysis, and ultrasonic fragmentation of DNA. 40 μL of His-tagged magnetic beads were added to the supernatant and rotated overnight at a low speed at 4° C. The beads were washed the next day, and protein complexes were eluted and de-crosslinked, followed by DNA purification using the QIAquick PCR purification kit (Germany). Primers were designed according to the promoter region of the ZmVIT2.1, and the relative abundance of a fragment with the MBS element (F1) and a fragment without the MBS element (F2, as the negative control) were analyzed by ChIP-qPCR analysis.

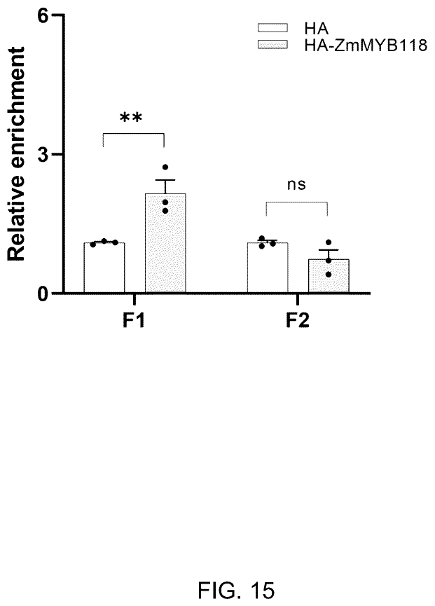

ChIP-qPCR results indicate that compared to the vector control, expression of the ZmMYB118 protein significantly enhances the relative enrichment of the MBS-containing promoter sequence of ZmVIT2.1, while there is no noticeable difference in the promoter region without the MBS element, indicating that the ZmMYB118 protein can bind to the MBS element in the promoter region of the ZmVIT2.1 in vivo (as shown in FIG. 15).

### (4) Dual-Luciferase Reporter Assay

A nucleotide sequence encoding the ZmMYB118 protein and a 1,970-bp upstream promoter sequence of ZmVIT2.1 (as shown in SEQ ID NO: 5) were cloned into the pGreen 62-SK vector and the pGreen II 0800-LUC vector, respectively, to generate 62-SK-ZmMYB118 and ProZmVIT2.1-LUC recombinant vectors, which were then verified by sequencing. Single colonies were selected and cultured, and plasmids were extracted and then transformed into Agrobacterium tumefaciens GV3101 containing pSOUP19. The 62-SK-ZmMYB118 Agrobacterium and the ProZmVIT2.1-LUC Agrobacterium were mixed at a ratio of 9:1 and co-transformed and infiltrated to Nicotiana benthamiana leaves. As a negative control, pGreen 62-SK empty vector and ProZmVIT2.1-LUC were co-transformed and infiltrated to Nicotiana benthamiana leaves and incubated for 48 hours. The dual-luciferase activity was measured using the GLO-MAX 20/20 luminometer (Promega). The activities of firefly luciferase (LUC) and Renilla luciferase (REN) were measured by a GLO-MAX 20/20 luminescence detector (Promega) with reference to the dual luciferase kit (Promega), and a LUC/REN ratio was calculated. The LUC/REN ratio of the negative control was standardized to 1.

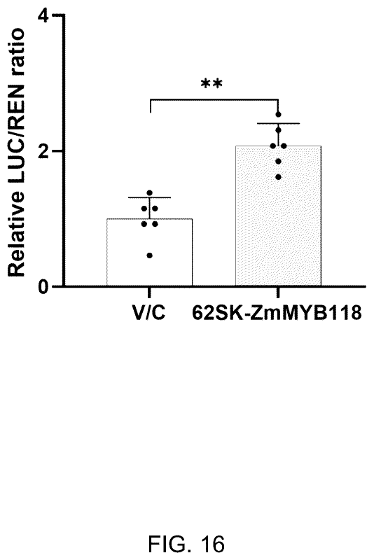

To verify whether ZmMYB118 regulates the expression of ZmVIT2.1 in plants, the dual-luciferase reporter assay was performed using the tobacco transient transfection assay. The results indicate that the LUC/REN ratio of 62-SK-ZmMYB118 is significantly higher than that of the negative control, indicating that ZmMYB118 activates ZmVIT2.1 expression (as shown in FIG. 16).

### Example 6: Heterologous Functional Verification of ZmVIT2.1 in Yeast

A nucleotide sequence of ZmVIT2.1 was cloned into a pDR195 vector. After sequencing confirmation, a single colony was selected and cultured, and plasmids were extracted. The yeast strain DEY1453 with deletions of both fet3 and fet4 was inoculated into the YPD liquid medium and shaking-cultured, then inoculated into 250 mL fresh YPD medium and shaking-cultured until the OD600 reached 0.4-0.6. The yeast cells were harvested by centrifugation at room temperature, washed with ultrapure water, and resuspended in 1.5 mL of 1×TE/LiAc. 10 μL of salmon sperm DNA, 150 ng of the corresponding plasmid, 100 μL of yeast competent cells, and 600 μL of PEG/TE/LiAc were added to a 1.5 mL centrifuge tube to obtain a mixture, and the mixture was incubated, followed by adding DMSO to stop the reaction. After centrifuging at room temperature and discarding the supernatant, yeast cells were resuspended in 200 μL of 1×TE buffer, and spread on the SD/−Ura solid medium and incubated at 30° C. for 2-4 days. Single colonies were selected and cultured in the SD/−Ura liquid medium until the OD600 reached 1. The bacterial liquid was then serially diluted to concentration gradients of 1, 10⁻¹, 10⁻², 10⁻³, and 10⁻⁴ based on cell density, and the diluted bacterial aliquots were spotted onto the SD/−Ura medium and SD/−Ura medium containing a ferrous-iron chelator of bathophenanthroline disulfonic acid disodium salt (BPDS) and incubated at 30° C. for 2-4 days to observe and record yeast growth. Yeasts transformed with empty vector pDR195 and Arabidopsis VIT1-pDR195 were used as negative and positive controls, respectively.

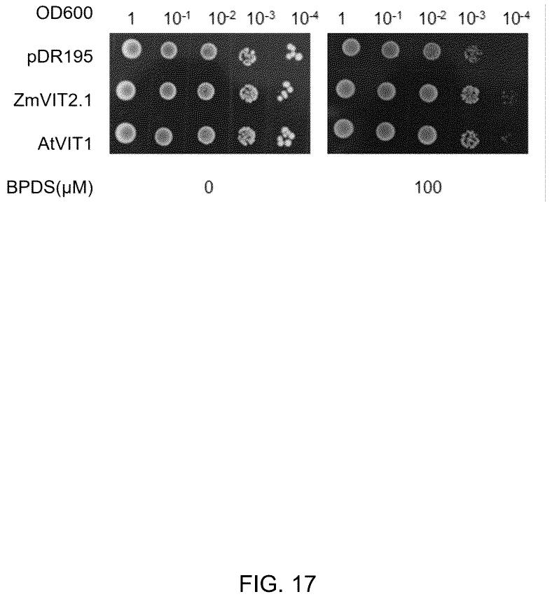

The results indicate that the growth of yeast strains transformed with the ZmVIT2.1 plasmid is better than that of the negative control transformed with the empty plasmid pDR195 under the condition of adding 100 μM BPDS, indicating that ZmVIT2.1 has the ability to transport ferrous iron (as shown in FIG. 17).

In summary, these findings indicate that the ZmCCD8 gene regulates the binding of ZmMYB118 to the promoter region of ZmVIT2.1 to activate an expression of the iron transporter, thereby contributing to iron accumulation in maize leaves and kernels.

The basic concepts have been described above, apparently, in detail, as described above, and do not constitute limitations of the disclosure. Although there is no clear explanation here, those skilled in the art may make various modifications, improvements, and corrections of the present disclosure. This type of modifications, improvements, and corrections are recommended in the present disclosure, so the modifications, improvements, and corrections remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

Furthermore, the present disclosure uses specific terms to describe the embodiments of the present disclosure. For example, “an embodiment”, “one embodiment”, and/or “some embodiments” refer to a particular feature, structure, or characteristic related to at least one embodiment of the present disclosure. Therefore, it should be emphasized and noted that “an embodiment” or “one embodiment” or “an alternative embodiment” mentioned in different locations of the present disclosure more than once does not necessarily refer to the same embodiment. In addition, some features, structures, or characteristics in one or more embodiments of the present disclosure can be appropriately combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, the present disclosure does not mean that the subject matter of the present disclosure object requires more features than the features mentioned in the claims. Rather, the claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the terms “about”, “approximate”, or “substantially”. Unless otherwise stated, “about”, “approximate”, or “substantially” may indicate ±20% variations of the value it describes. Accordingly, in some embodiments, the numerical parameters used in the disclosure and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other materials cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to these embodiments precisely shown and described.

What is claimed is:

1. A use of a ZmCCD8 gene in maize breeding, comprising:
overexpressing the ZmCCD8 gene in maize plants through breeding to increase iron content in maize leaves and/or kernels, wherein the ZmCCD8 gene has a nucleotide sequence as shown in SEQ ID NO: 1.

2. The use of claim 1, wherein
the ZmCCD8 gene encodes a protein having an amino acid sequence as shown in SEQ ID NO: 2.

3. The use of claim 1, wherein
overexpressing the ZmCCD8 gene in the maize plants increases the iron content in ear leaves.

4. The use of claim 1, wherein
overexpressing the ZmCCD8 gene in the maize plants increases the iron content in kernels per ear.

5. A use of a ZmCCD8 gene in regulating the interaction between a ZmMYB118 protein and a promoter of ZmVIT2.1, comprising:
overexpressing the ZmCCD8 gene in maize plants through breeding to regulate the interaction between the ZmMYB118 protein and the promoter of ZmVIT2.1;
wherein the ZmCCD8 gene has a nucleotide sequence as shown in SEQ ID NO: 1, the ZmMYB118 protein is encoded by a nucleotide sequence as shown in Zm00001d032024 from a Phytozome database, and the promoter of ZmVIT2.1 has a nucleotide sequence as shown in Zm00001d005715 from the Phytozome database.

6. A method for maize breeding, comprising:
overexpressing a ZmCCD8 gene in maize plants to increase iron content in maize leaves and/or kernels; and obtaining a maize inbred line overexpressing the ZmCCD8 gene;
wherein the ZmCCD8 gene has a nucleotide sequence as shown in SEQ ID NO: 1.


SEQUENCE LISTING (5 sequences)

SEQ ID NO: 1 - DNA, 1719 bp, Zea mays
  source 1..1719: mol_type = genomic DNA
atgtctcccactatggcttcgtcgttgtgcgtatttgcagcgatgtctggcgccagcggcaggccgtcggccggtggctcggcggtaccgggccgtctgtccagcagcacacaggggggcaagggaaagcgggccgtggtgcagccgctcgcggctagcgtggtgacggagacgccagcgccggccgtagctccggctcggcccgtcgtcgacgccccgccgcgccgccgtgggggccgcggcaccgtcgagcacgcggcgtggaagagcgtccggcaggagaggtgggagggggcgctggagctggagggagagctgccgctctggctggatggcacctacctgaggaacgggccgggcctgtggaacctgggcgactacggcttccggcacctgttcgacggctacgccacgctggtccgcgtgtcgttccgcgacgggcaggcggtgggcgcgcaccggcagatcgagtcggaggcgtacaaggcggcgcgcgcgcacggcaaggtgtgctaccgcgagttctcggaggtgcccaaggcggaggggttcctctcccacgtgggccagctcgccaccctcttctcgggctcctcgctcaccgacaactccaacacgggcgtcgtcaggctcggcgacggccgcgtcctctgcctgacagagaccatcaagggctccatcgtggtcgacccggacacgctcgacaccatcggcaagttcgagtacacggacaggctgggcggcctcatccactcggcgcaccccatcgtgacggacaccgagttctggacgctcatcccggacctcatccgcccgggctactcggtggtgaggatggacgccgggaccaacgagcggcggttcgtcggcagggtggactgtcggggcgggccggcgcccgggtgggtgcactcgttccccatcaccgaccactacgtggtggtgccggagatgccgctccggtactgcgccaggaacctcctccgcgcggagcccacgccgctgtacaagttcgagtggcacctcgagtccggcagctacatgcacgtcatgtgcaaggctagcggcagggtcgtggcgaccgtggaggtgccgccgttcgtcacgttccacttcatcaacgcgtacgaggagaaggacgacgagggccgcgtcaccgcgatcatcgccgactgctgcgagcacaacgccaacacctccatcctcgacaagctccggctccagaacctgcgctcttccaccggccaggacgtcctccccgacgccagggtgggccgcttcaggatcccgctggacgggagcccgttcggcgagctggagccggcgctggacccggaccagcacggccgcgggatggacatgtgcagcatcaacccggcccacgtcggcaagaagtaccggtacgcctacgcctgcggagcccaccggccgtgcaacttccccaacaccctcaccaagatcgacctggtggagaagacggccaagaactggtacgaggagggcgccgtgccgtcagagcccttcttcgtgccgcgccccggcgccgtggaggaggacgacggcgtcgcgatctcgatggtgagcgccaaggacggatcggcctacgcgttggtgctggacgccaagacgttccacgagatcgcgcgggccaagttcccgtacgcgatgccctacggcttgcactgctgctgggtgcctaggagtacctcagacgcgtag

SEQ ID NO: 2 - protein, 572 aa, Zea mays
MSPTMASSLCVFAAMSGASGRPSAGGSAVPGRLSSSTQGGKGKRAVVQPLAASVVTETPAPAVAPARPVVDAPPRRRGGRGTVEHAAWKSVRQERWEGALELEGELPLWLDGTYLRNGPGLWNLGDYGFRHLFDGYATLVRVSFRDGQAVGAHRQIESEAYKAARAHGKVCYREFSEVPKAEGFLSHVGQLATLFSGSSLTDNSNTGVVRLGDGRVLCLTETIKGSIVVDPDTLDTIGKFEYTDRLGGLIHSAHPIVTDTEFWTLIPDLIRPGYSVVRMDAGTNERRFVGRVDCRGGPAPGWVHSFPITDHYVVVPEMPLRYCARNLLRAEPTPLYKFEWHLESGSYMHVMCKASGRVVATVEVPPFVTFHFINAYEEKDDEGRVTAIIADCCEHNANTSILDKLRLQNLRSSTGQDVLPDARVGRFRIPLDGSPFGELEPALDPDQHGRGMDMCSINPAHVGKKYRYAYACGAHRPCNFPNTLTKIDLVEKTAKNWYEEGAVPSEPFFVPRPGAVEEDDGVAISMVSAKDGSAYALVLDAKTFHEIARAKFPYAMPYGLHCCWVPRSTSDA

SEQ ID NO: 3 - DNA, 40 bp, synthetic construct
  source 1..40: mol_type = other DNA
ggtcgtcagctatagataactgctgacggtcctttgccga

SEQ ID NO: 4 - DNA, 40 bp, synthetic construct
  source 1..40: mol_type = other DNA
tcggcaaaggaccgtcagcagttatctatagctgacgacc

SEQ ID NO: 5 - DNA, 1970 bp, Zea mays
  source 1..1970: mol_type = genomic DNA
tgaagtaaagtaaacaaaacggatgtgatcatgcttaagctagcttccgttagtgactattttgctagaggctctaaaaggtattccgatattgcactttgaaagtatcaaaatttgatagagaaaattagtgactagtgttagatcattggggtagatcagtggggtgatctcaattcatttgtgtaattggtgtgttataaccactagatttaaatcataaatagattataccgttagtggggggtcacacttgtggtggaggatgtagatctgattgccatcacgttcgtcgatgaagatctgcactagggcagcaacggtcggggaagcgaggttggtggggtgaagactgacggcggtttctcgtcactggtcgatcggtggtttatgtttttgtcggtggggaatacggctcaagtgtgacactcgacaaagaaagttcggcgaactgtacatcggcaatggtttctttgctgactactttttatcgggtactcggcaaagtctttgccgagtgtcactcgatactcggcaacgaaaagccgtcgtcacggtgccaggtaacggcgctcggcaaagataacggtcgtcagctatagataactgctgacggtcctttgccgagcgtcgacgttcgccgagtgtttggcactcggcatagcggtctttgtcgagtgtctgcatatgccgagagtcctgctctcggcaaatgtggtcgttactgagagtaggactgtgccgagtgcggcactcaacaaagacttttttgccgagtgtccgacaaaaagcaatcggcaaagcatcgagcactcggcaatgagacggatttcggtagtgagtgttaaaccattggggtagatcagtgggatgatctctattcatttgtgtaattgttgtgtcctaaccactagatttaaataaccactagaattaaatcataaatagattataccgtcggtgggggttgcacttggggtggaggctgccatcaggttcgtcgatgaagatctgctctagggcagcaagagtcggggaagtgaggttgatggcgatgaagaccgacggtggcttctcgtcactagtcgatcggtagtttatgtttttgtcggtgaggaatacggctcaagagaaccgtcggacttcacctctttgtatggcccagtgtgacagggatccattaaccacacgtaaggttgggtgtcctcgctcaggacgcaaatcaaaggccgaagatagccgttgggtcattttggtggagatcattccaacagctagacgaaaacaggtacaataggttatggtgtgtgtgttttttacagaaaccatgagctcattcctactacactaaacttctttttaaggtacatggtatatatgtcaaattaaaatgacatctatagtggattttgtatttgaagtcagctgccaaagagaaatcagcgtagtaaaccagaaattcacactccacgtgattggttgctcggatagctacacgcaggcccgttgtatgcattgaccgtgaatgggcatacgattgattacctgtactatacaggtgcagcctgcatgcacgaaagcaggtcaagcgtttttccaaccctctgcctgcacgcgtggagatgctggggaggggcttcccggatcgcccaggccagcgtgagcatggctcccgttggcaccacaaccaatcacgcccactctggtttgtgaagctacccactgcaaaagccacaaaagcagcagcaaaaccaccaccccatgtccaagttggtcatttccgcccgtgctaacccctataaatcccccctcctagctctgcgttggcctcaccaacacagaaaacctcgcagcagcagcagcaggatcatagcacaaacacgaaggttaatcaagcgagcgcttagctaataagcgcgcggtcttctttttcctcgagagtgagtgagagtattgaggcgcgagaagag